(19) United States
(12) Patent Application Publication
(10) Pub. No.: US 2015/0148538 A1
(43) Pub. Date: May 28, 2015

(54) USE OF AN H4 AGONIST MOLECULE TO TREAT ACUTE LEUKEMIA

(71) Applicant: Gaetan TERRASSE, SAINT VALLIER (FR)

(72) Inventor: Gaetan TERRASSE, SAINT VALLIER (FR)

(21) Appl. No.: 14/555,773

(22) Filed: Nov. 28, 2014

Related U.S. Application Data
(60) Provisional application No. 61/909,614, filed on Nov. 27, 2013.

(51) Int. Cl. C07D 498/04

(52) U.S. Cl. CPC C07D 498/04
USPC 546/90

(57) ABSTRACT

The present invention relates to the use of new chemical substances, the levogyre and dextrogyre enantiomers of (AMINO-7 TRIETHOXY-4,5,6 OXO-1 DIHYDRO-1,3 ISOBENZOFURANNYL-3)-1 METHOXY-8 METHYL-2METHYLENEDIOXY-6,7 TETRAHYDRO-,2,3,4 ISOQUINOLEINE or tritoqualine, to treat acute myeloid or lymphoid leukemia, with the exception of type B leukemia.

### FIELD OF THE INVENTION

The present invention relates to the use of chemical substances, the levogyre and dextrogyre enantiomers of (AMINO-7 TRIETHOXY-4,5,6 OXO-1 DIHYDRO-1,3 ISOBENZOFURANNYL-3)-1 METHOXY-8 METHYL-2METHYLENEDIOXY-6,7 TETRAHYDRO-,2,3,4 ISOQUINOLEINE or tritoqualine to treat acute leukemia.

### BACKGROUND OF THE INVENTION

Acute Leukemias (AL) are malignant hemopathies, characterized by two aspects:
The clonal proliferation of abnormal myeloid or lymphoid precursors (cells with little differentiation);
Altered hematopoiesis.

The blasts of the different forms of Leukemia (AL) have fundamental oncogenic properties:
blockage of differentiation;
uncontrolled cell proliferation.

These are diseases which, if untreated, are life-threatening and constitute a diagnostic and therapeutic emergency (HAS, November 2011).

Despite recent therapeutic progress and new developed care strategies, some of these diseases remain resistant to treatment and have rapid and fatal relapses.

In Acute Lymphoblastic Leukemia (ALL) and Acute Myeloid Leukemia (AML), age is a deciding factor in the prognosis, due to high-risk forms and an increasingly mediocre tolerance for chemotherapy based on aging (SFH, 2009 Referentiel).

Between 1980 and 2005, the incidence of acute leukemia (AL) increased regularly (0.9%); the hypothesis submitted by experts is that this is more related to acute myeloid leukemia (AML), in particular secondary, due to aging of the population.

However, as emphasized by the Institut de veille Sanitaire [Health Watch Institute], the lack of data on sub-groups of acute leukemia (AL) in France makes it impossible to confirm this (Institut de Veille Sanitaire).

Depending on the authors (HAS, November 2011), acute leukemia (AL) represents 1 to 2% of all cancers and is ranked 21st among cancers (Institut Veille sanitaire), but 12th in terms of mortality. There is therefore still a true need today.

In Europe, the annual incidence of acute myeloid leukemia (AML) is 2.7/100,000, and that of acute lymphoblastic leukemia (ALL) is 1.5/100,000 (O'donnell. Acute Leukemia: 2011 Cancer network-home of the journal Oncology), with a mortality rate of 2/100,000 per year.

In France in 2005, acute leukemia (AL) represented 3082 cases and 2700 deaths; the standardized incidence rate for acute leukemia (AL) was 4.5 in men and 3.5 in women, that cancer being ranked 16^{th} in number of deaths (Institut de Veille sanitaire).

In France in 2010, the number of incident cases had evolved slightly and represented 3500 cases (HAS, November 2011).

In the United States, the incidence of acute myeloid leukemia (AML) is 3.4/100,000 and acute lymphoblastic leukemia (ALL) is 1.5/100,000. The numbers estimated by the NCI in 2013 reached 17,018 new cases and 10,900 deaths (NCI-US National Cancer Institute).

Acute myeloid or myeloblastic leukemia (AML) represents 70% of cases of acute leukemia (AL) for which the average age of occurrence is 65-70 years.

The incidence of acute myeloid leukemia (AML) increases with age, which makes it a major issue in current hematology.

Characteristics of acute myeloid leukemia (AML):
The survival rate varies based on the age at which the disease appears: in young adults, it is 30-40% at 3 years, but only 10-20% in subjects over 50 years of age (Ferrara F Lancet 2013—Appelbaum Blood 2006).
The likelihood of 5-year survival in a young adult (15-60 years) with a relapse after a 1^{st} complete remission is defined using a risk index (Döhner H Blood 2010).

The distribution of patients suffering from acute myeloid leukemia (AML) based on risk type:
Favorable risk=9% of AML
Intermediate risk=25%
Unfavorable risk=66%

The likelihood of survival, for 66% of subjects under 60 years (unfavorable risk), is only 16% at 1 year and 4% at 5 years (DÖHNER H Blood 2010). The medical risk is therefore high, including in young patients.

The complete remission (CR) levels are 70% in those under 60 years (course) and only 40% in those over 60 years (Fathi Curr Oncol Rep 2009).

The relapse rates after a first complete remission (CR1) are extremely high, although variable from 30 to 80% and observed in most cases within 3 years after diagnosis. In general, the prognosis is poor and the therapeutic options are unsatisfactory at the time of the relapse (Döhner H Blood 2010).

In the case of relapse after a first complete remission (CR1), the chances of obtaining that second remission (CR2) depend on the length of the first remission (CR1) (Mangan J K, Ther Adv Hematol 2011):
If the first remission (CR1)>1 year, the second remission (CR2) is obtained in 60% of cases;
If the first remission (CR1)>6 months, the second remission CR2 will be obtained in less than 20% of cases.

Acute lymphoblastic leukemia (ALL) represents 30% of cases of acute leukemia (AL), 80% of which are seen in children.

In adults, the incidence peak is around 50 years (Faderi Cancer 2010), but when it affects an adult, the complete remission rates are low compared to those observed in children (Narayanan Crit. Rev. Oncol. Hematol. 2012). Frequent relapses (>45%) and survival rates (45-75%) are lower than in children.

In the long term, the survival rate without relapse after a first complete remission is greater than 40% (Faderi Cancer 2010)

In acute lymphoblastic leukemia (ALL), the survival and complete remission rates during treatment with relapse after a first remission are indicated below. It must be noted that 21% of patients pass away before a response to treatment is obtained and that among refractory subjects, during first induction, only 34% obtain complete remission (CR) (Thomas D A, Cancer, 1999).

The overall median duration of the complete remission (CR2) is only 6 months, and the median survival time is 5 months; only 24% of patients are still alive at 1 year, and 3% at five years.

Overall, in acute leukemia (AL) cases in adults, the survival rate 5 years after the first relapse is approximately 10% (Forman Blood 2013).

In France, the mortality rate (Networld standardized for 100,000 inhabitants) is 2.8 in men and 1.9 in women (Institut de veille sanitaire).

In the US, the mortality rate is 2.8/10,000 for acute myeloid leukemia (AML) and 0.5/100,000 for acute lymphoblastic leukemia (ALL) (US National Cancer Institute).

There is therefore still a real medical need today for new and innovative therapeutic strategies making it possible to optimize therapeutic care for ALL and AML in adults.

Treatments for acute leukemia (AL) are in particular based on long and intensive sequential polychemotherapy, adapted to age and done in several stages:
Induction chemotherapy targets remission and is accompanied by +/−salvage courses;
Then consolidation chemotherapy, the number of courses of which varies based on the type of AL and prognostic factors;
And next, still depending on these prognostic factors and the type of acute leukemia (AL), there is a discussion of prolonged maintenance chemotherapy or a stem cell allograft or intensification with or without autologous stem cells (Ron Ram Cancer 2010, Marks D Hematology 2010, SFR referential 2009, HAS 2011.

The induction polychemotherapy combines different products whose tolerance remains mediocre and the side effects of which are severe. Each polychemotherapy cycle is systematically followed by a prolonged aplasia with 4 to 6 weeks of hospitalization in a protected unit.

The treatment for acute myeloid leukemia (AML) is different based on the patient's age and general condition:
In those under the age of 60 years, the aim is curative, with intensive chemotherapy lasting 6 months, the reference protocol of which combines cytarabine and anthracycline. Based on the cytogenetic risk after the induction phase, a consolidation phase by chemotherapy or hematopoietic stem cell graft will be done to prevent relapse. Complete remission is a prerequisite for long-term survival.
In those over the age of 60 years, it is essential to identify the unfavorable prognostic elements (fragility, comorbidity, unfavorable cytogenetics).

Patients with an ECOG score greater than 2 and over the age of 80 years, signs of infection, comorbidity and/or unfavorable cytogenetics cannot receive standard chemotherapy; in Europe and the US, they will then be offered small doses of cytarabine or palliative treatment using hydroxyurea or 6-mercatopurine associated with asymptomatic treatment as a 1^{st} line.

The most commonly used treatments are anthracyclines, epipodophyllotoxins, methotrexate, thiopurines, cytarabine and alkylant agents such as cyclophosphamide.

All of these treatments are toxic and cause many side effects.

These treatments aim to block the development of cancerous cells by killing them or limiting their division, but at this time they act on all cells, including healthy cells, therefore causing major side effects that increase morbidity and mortality.

As an example, in acute myeloid leukemia (AML), in young adults (18-60 years), the induction phase combines 3 days of anthracycline (ex-daunorubicin—DNR) or idarubicin and 7 days of cytarabine; for subjects over 60 years, the doses may be adapted, and the therapeutic strategy is then customized.

Relapses:
The risk of relapse (reappearance after complete remission of the blasts) nevertheless remain high, depending on the form and/or age. The majority of acute myeloid leukemia (AML) cases relapse, including 40 to 50% of those with a risk classified as “favorable” (Tara et lin Oncology 2012).

Refractory forms:
Lack of response after 2 chemotherapy cycles (induction and salvage), forms with very high risk of later failure (20 to 30% of AML cases) (Lin et Levy 2012).

Ineligible patients:

Some patients, including elderly patients, are not eligible for intensive chemotherapy and are offered palliative care—the purpose of which is quality of life—anti-infectives, or participation in clinical trials.

However, this issue of tolerance is widely discussed in the literature and remains the sticking point for the activity of polychemotherapies, the optimal effective doses of which sometimes cannot be administered (Ziogas D C, Clin Ther, 2011; David C Curr. opin. Support. Palliat. Care 2009, Cullen M Br. Cancer 2009, Appelbaum blood 2006).

In acute lymphoblastic leukemia (ALM) in adults, the optimal initial treatment is not established (Morris K leuk lymphoma 2011). There are several induction treatments, hyper-CVAD (Cyclophosphamide, vincristine, doxorubicin and dexamethasone) yield the highest complete remission rates (Marks Hematology 2010), but in adults over 60 years, the long-term survival rate remains low (<20%).

It is commonly recognized that post-remission relapse is the main cause of death in these patients. Many pass away quickly after the relapse, and many do not reach the 2^{nd} complete remission (Marks, American Society of hematology 2010).

In the event of relapse, the conventional treatments combine vincristine, steroids, and anthracyclines; asparaginase and methotrexate or high doses of cytarabine, and yield poor results to date (Ram Cancer 2010).

The strategies under development in acute myeloid leukemia (AML) (Tara Lin 2012) aim or have tried to achieve the following objectives:
Improve the response to induction treatment by intensifying chemotherapy doses (G Juliusson Blood 2009), or consolidation courses with high doses or combination with new molecules (immunosuppressant, anti-CD 33);
Decrease the toxicity of induction treatments, for example with hypomethylating agents (AZA), immunomodulators (lenalidomide);
Prolong remissions (Ara-C, etoposide, IL-2 combined).
Establish new therapeutic strategies when relapses occur or in refractory acute myeloid leukemia (AML) (FLT3 inhibitors, clofarabine).

The more particularly interesting molecules, which are currently undergoing clinical trials in acute myeloid leukemia (AML), are gemtuzumab ozogamicin (anti-CD33 humanized antibody—Pfizer), FLT3—selective tyrosine kinase inhibitors (midostaurin, lestaurtinib, sunitinib) and to do mentally agents (azacitidine and decitabine) (Döhner H Blood 2010) seeking targeted therapy on the underlying oncogenic mechanisms.

Nevertheless, some of these have a high toxicity (Gemtuzumab ozogamicin—anti CD33) or an efficacy that, for the moment, does not meet expectations (FLT3 inhibitors, ex midostaurin (A T. FATHI, B A. CHABNER: The Oncologist 2011).

Strategies under development in acute lymphoblastic leukemia (ALL):
Intensification: the “Hyper-CVAD” protocol (hyperfractionated cyclophosphamide, doxorubicin, vincristine, methotrexate, cytarabine) seems to have made it possible to improve complete remission rates, but with high toxicity causing premature treatment stoppages, for example resulting in attempts at early intensification of anthracycline doses, but which have not yielded improved results (Thomas D cancer 2010).
New molecules at early development stages (preclinical/in vitro) such as an Akt inhibitor (MK226) or an interleukin 2 inducing the T-cell kinase inhibitor that may potentially act on type T ALL (Simoni Leukemia 2012, Guo W Mol. pharmacol 2012) or a new generation of JAK inhibitor (type II) that can act on B lymphocyte precursors, or anti-CD 19 or CD 22 in relapsing and refractory forms of ALL.

Products such as nelarabine (LALT) that have a dose-dependent neurotoxicity are also in development, as well as clofarabine, the results of which remain mixed.

New developments are turning to therapeutic consolidation strategies evaluating moderate intensity doses of chemotherapy in order to increase the 1-year survival rate by limiting risks of treatment-related death (Faderi Cancer 2010).

In this indication, new molecules are therefore desirable, as well as new care strategies (Faderi Cancer 2010).

Despite the many therapeutic classes that are used, and although acute leukemia patient survival has increased, the results of treatments are still largely insufficient.

Tritoqualine is a chemical substance that has been known for many years, and is used as an antihistamine. Its manufacture is described in French patent FR 1,295,309.

Tritoqualine is known for its anti-allergic activity through its histidine decarboxylase inhibiting action.

This activity is, however, very low and does not explain the many properties that it has with respect to various clinical symptoms: rhinitis, urticaria, eczema, mastocytosis.

The applicant, and others, have demonstrated that tritoqualine had a very significant activity on a new receptor, the histamine H4 receptor.

This activity of tritoqualine on the H4 receptor was demonstrated in an American patent application US2010144718A1 “TREATMENT OF DISEASES MODULATED BY A H4 RECEPTOR AGONIST”. This patent application does not, however, describe the activity of tritoqualine in acute leukemia.

Another patent application WO2008006974A2 on H4 agonists describes the use of these products in the protection of hematopoiesis precursors as part of chemotherapy.

It will be noted that to date, neither the aforementioned patent applications nor any other patents or documents have shown the action of histamine H4 agonists on acute leukemia.

Commercial tritoqualine assumes the form of a white powder that is very sensitive to light, which breaks it down into Cotarnine and phthalic acid.

Commercial tritoqualine (called Hypostamine) assumes the form of a tablet with a concentration of 100 mg per tablet.

The following studies have been conducted with a purified sample of commercial tritoqualine.

### SUMMARY OF THE INVENTION

We dilute the tritoqualine in dimethyl sulfoxide, also called DMSO. This operation is necessary due to the low solubility of tritoqualine in water, including with fetal calf serum added.

To obtain it at 10⁻²M, we perform the following calculation:
For tritoqualine:
   Quantity of DMSO in μL=(quantity of tritoqualine weighed in mg*1000)/5, the molecular mass of tritoqualine being 500.

Subsequently, we dilute it, to use from 10⁻⁴M to 10⁻⁵M, in a culture medium with 10 or 20% fetal calf serum. This dilution corresponds to doses used in humans (weight between 50 and 70 kg) from 250 mg to 3000 mg.

Tritoqualine is used in the experiments preferably a dose from 500 mg to 1000 mg after adjusting concentrations in the culture medium.

Under these conditions, the astonishing properties of commercial tritoqualine on leukemia cells have been shown, while that product has a very low toxicity, which appears to be paradoxical.

The action mechanism is related to physiological blocking of cell proliferation, without triggering toxic cell death. In leukemia, the uncontrolled proliferation of malignant lines is responsible for smothering normal lines.

This physiological mechanism blocking the proliferation of malignant lines is a new action mechanism in leukemia. It makes it possible to reduce tumor mass and complete the treatment with traditional chemotherapies.

### BRIEF DESCRIPTION OF THE DRAWINGS

FIG. 1 illustrates the presence of asymmetrical carbons, which are denoted A and B.

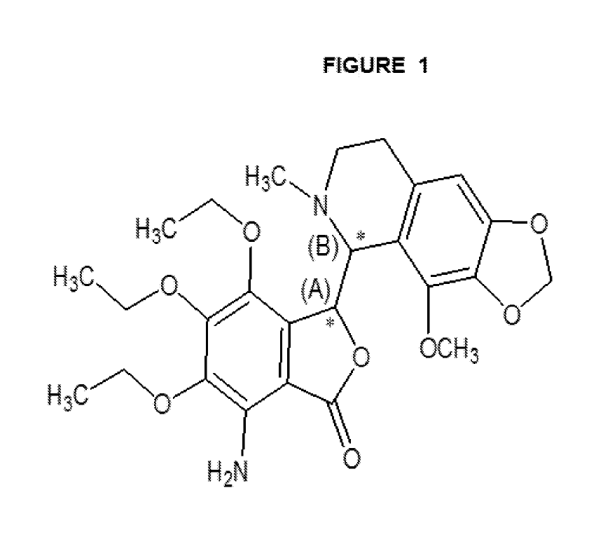

FIG. 2 illustrates the form of the D1 isomer.

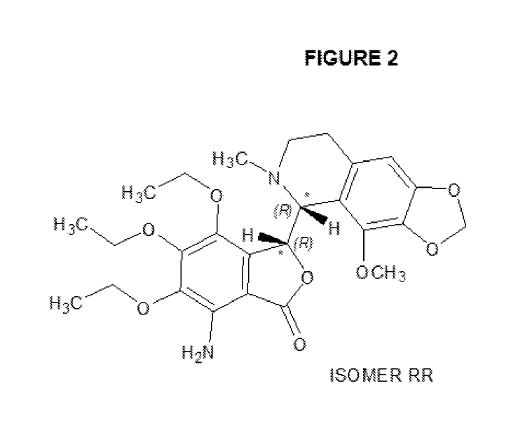

FIG. 3 illustrates the form of the D2 isomer.

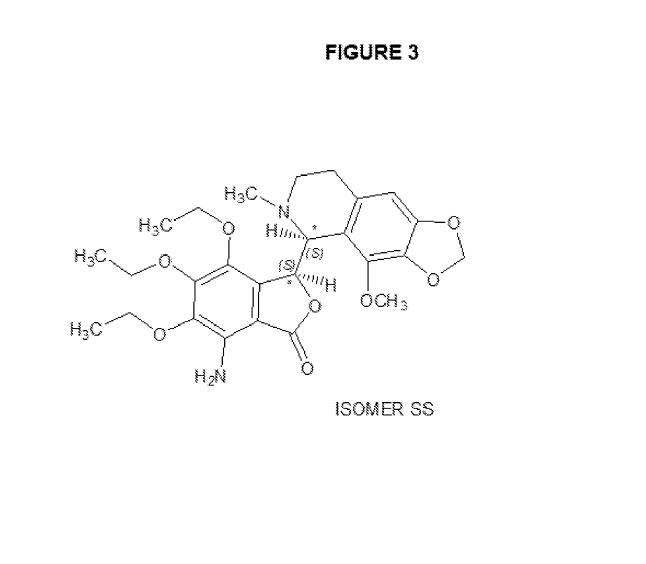

FIG. 4 shows the inhibition experiment for the myeloid line using tritoqualine, compared to Clobenpropit.

FIG. 5 shows that tritoqualine inhibits the Pre T line, but not the Pro B line.

### DETAILED DESCRIPTION OF THE INVENTION

The examples below will show the impact of tritoqualine on the proliferation of malignant lines in the 2 main lines, i.e., the myeloid line and the lymphoid line.

However, it appears that the lymphoid B lines are not sensitive to the action of tritoqualine.

The latter line apparently lacks histamine H4 receptors, which could explain the inefficacy of tritoqualine on that line.

### EXAMPLE 1

The myeloid line is represented by the following clonal malignant cells: the HL60 line and the TF1 line. TF1 constitutively expresses the histamine H4 receptor and proliferates in response to GMCSF.

The HL 60 line also expresses the histamine H4 receptor, but more significantly than on TF1 lines.

This difference could explain the difference in inhibition percentage of malignant cells.

Clobenpropit (CB, H4 agonist) inhibits the proliferation of this line at the traditionally used dose of 10⁻⁵M.

Tritoqualine, a mixture of 2 enantiomers, has been tested on this line between 10⁻⁵ and 10⁻⁷M with or without CB 10⁻⁵M addition.

The TF1 line and the HL60 line are represented by inoculates of 100,000 cells in one milliliter incubated for 3 days with GM-CSF (10 ng/ml).

The reading is done on the third day and the number of cells in the proliferation phase is measured. The ratio between the cells in the proliferation phase and the cells that do not proliferate yields the percentage of cells in inhibition. Normally, all of the cells are in the proliferation phase when the cells are stimulated with GMCSF.

### EXAMPLE 2

The lymphoid line is represented by the following clonal malignant cells: the Pre T line and the Pro B line. The cells are cultured using the same technique as the myeloid lines.

The reading is done on the third day, and the number of cells in the proliferation phase is measured. The ratio between the cells in the proliferation phase and the cells that are not proliferating yields the percentage of cells in inhibition. Normally, all of the cells are in the proliferation phase when the cells are stimulated with GMCSF.

The results show that tritoqualine and Clobenpropit block the proliferation of the Pre T cells, but not that of the Pro B cells.

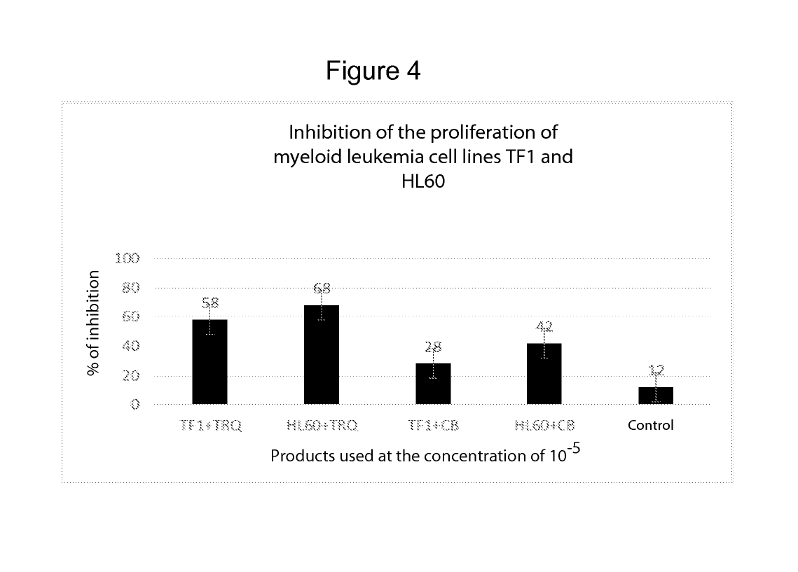

FIG. 4 shows the results, which reveal a strong inhibition of the proliferation of TF1 and HL60 cells.

The results are expressed in proliferation inhibition % (n=3): The result is the Mean of the three tests performed.

The results are expressed in estimated proliferation inhibition % by cell count or by rock proliferation test. This test measures the incorporation of a dye when the cells are proliferating (XTT test).

At 10⁻⁵, tritoqualine inhibits approximately 58% of TF1 cells and 68% of HL60 cells. This result is surprising, since it is more powerful than that of Clobenpropit, even though the latter is a more powerful histamine H4 agonist than tritoqualine (affinity 10⁻⁹ versus 10⁻⁶).

The use of a histamine H4 antagonist in turn inhibits the effect of tritoqualine. This appears to indicate that the H4 agonist effect is indeed responsible for the activity of tritoqualine.

This indicates that tritoqualine has a powerful inhibiting activity regarding the proliferation of myeloid leukemia cells (TF1 and HL 60 representing the myeloid line).

The cell analysis shows that the cells are either in G0 phase, or G1 phase.

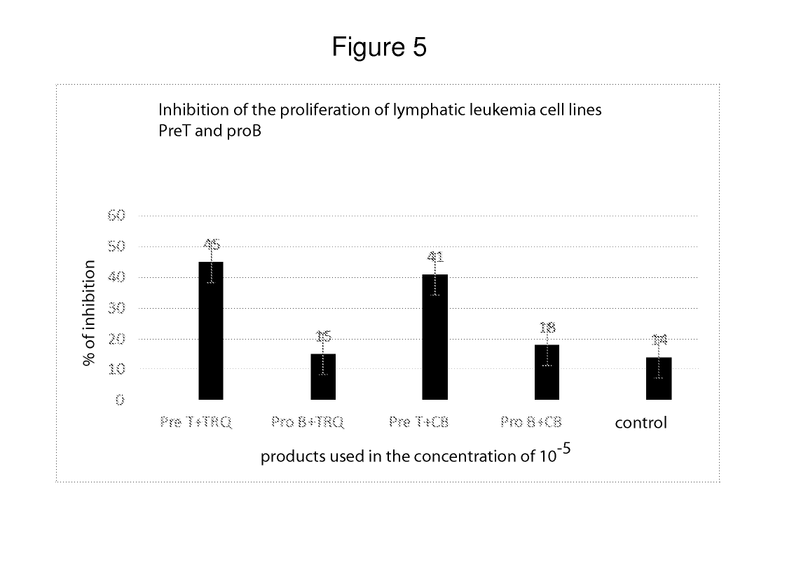

FIG. 5 shows the results that reveal a strong inhibition of the proliferation of Pre T cells, but not Pro B cells.

The results are expressed in proliferation inhibition % (n=3): The result is the Mean of the three tests done.

The results are expressed in estimated proliferation inhibition % by cell count or by rock proliferation test. This test measures the incorporation of a dye when the cells are proliferating (XTT test).

At 10⁻⁵, tritoqualine inhibits approximately 45% of Pre T cells, but only 15% of Pro B cells. This result shows that in the B line, the proliferation inhibition is equal to the control without product. This indicates the lack of efficacy of tritoqualine on the B lines.

The H4 receptor research shows that the latter is missing from the B line.

Tritoqualine has 2 asymmetrical carbons, but the commercial form is a mixture of 2 enantiomers.

1. A substance having an agonist activity on the histamine H4 receptor to treat acute myeloid leukemia and type T lymphoid leukemia, with the exception of type B leukemia, characterized in that it is made up of an isomer or an isomer mixture of AMINO-7 TRIETHOXY-4,5,6 OXO-1 DIHYDRO-1,3 ISOBENZOFURANNYL-3)-1METHOXY-8 METHYL-2METHYLENEDIOXY-6,7 TETRAHYDRO,2,3,4 ISOQUINOLEINE, or tritoqualine.

2. The substance according to claim 1, characterized in that it inhibits the G0/G1 phase proliferation of myeloid leukemia cells and lymphoid cells, with the exception of type B leukemia cells, and in that it is non-toxic for normal hematopoietic stem cells.

3. The substance according to claim 1, characterized in that it is packaged in doses from 100 to 2000 mg.

4. The substance according to claim 1, characterized in that it is packaged in different galenic forms such as tablets, gel caps, gel, capsules, injectable solution.

5. The substance according to claim 2, characterized in that it is packaged in doses from 100 to 2000 mg.
